(19) United States
(12) Patent Application Publication
(10) Pub. No.: US 2011/0172704 A1
(43) Pub. Date: Jul. 14, 2011

(54) SOLDER FORMULATION AND USE IN TISSUE WELDING

(75) Inventors: Benjamin S. Bleier, Villanova, PA (US); Noam A. Cohen, Bala cynwyd, PA (US); James N. Palmer, Philadelphia, PA (US)

(73) Assignee: THE TRUSTEES OF THE UNIVERSITY OF PENNSYLVANIA, PHILADELPHIA, PA (US)

(21) Appl. No.: 13/119,659

(22) PCT Filed: Sep. 18, 2009
(86) PCT No.: PCT/US09/57419

§ 371 Date: Mar. 17, 2011

Related U.S. Application Data
(60) Provisional application No. 61/098,409, filed on Sep. 19, 2008.

(51) Int. Cl. A61B 17/00; A61K 38/38; A61P 17/02

(52) U.S. Cl. 606/213; 514/15.2

(57) ABSTRACT

Supersaturated gel formulations including a solution of chitosan, albumin, and a laser specific chromophore. Laser tissue welding methods using the gel formulations of the present invention are also described. In the methods, the gel formulation of the present invention is provided to a site for tissue repair and the laser specific chromophore within the gel is excited with a laser in order to fuse tissue by inducing protein denaturation. The gel formulations and laser tissue welding methods may be used, for example, to enable skull base repairs, aerodigestive endoscopic repairs, endoscopic endonasal surgical repairs, iatrogenic esophageal perforation repairs, laparoscopic abdominal surgical repairs, lung repairs, colon repairs, anastomosis of vessels, urologic/gynecologic endoscopic pelvic repairs, orofacial surgical repairs, dental replacement, skin closure, uterine closure and repairs after fibroidectomies and bladder surgery.

### BACKGROUND OF THE INVENTION

1. Field of Invention

The present invention is directed to a solder formulation and to methods of tissue welding employing the solder formulation. The solder formulation provides strong bonding which may be advantageous in a variety of applications involving tissue welding.

2. Brief Description of the Prior Art

As surgical procedures advance, a limiting factor has become the ability to reliably repair the complex defects created by theme approaches. Tissue defects arising in, for example, endoscopic skull base surgery are difficult to reliably repair. Current multilayer techniques have evolved from experience with endoscopic cerebrospinal fluid leak (CSF) repair. However a reliable method of isolating the sinonasal and intracranial compartments remains elusive and the patients who fail may be subjected to morbid interventions such as lumbar drain placement or open craniotomy. Laser tissue welding offers the ability to create durable endonasal tissue bonds and may provide a solution to this surgical dilemma.

Laser tissue welding (LTW) utilizes a biologic solder doped with a laser specific chromophore which fuses tissue edges through protein denaturation following laser exposure. Such welds may produce tissue bonds capable of withstanding pressures exceeding human intracranial pressure with negligible collateral thermal tissue damage. LTW can be performed endoscopically utilizing a fiber optic cable and is thus ideally suited for use at the skull base. The use of biologic solders has been shown to provide a non-immunogenic scaffold for wound healing which contrasts with the granulomatous inflammatory response that is typically seen with use of suture material. Kirsch A J, Miller M I, Hensle T W, Chang D T, Shabsigh R, Olsson C A, Connor JP, “Laser tissue soldering in urinary tract reconstruction: first human experience,” Urology. 1995 August;46(2):261-6. Also, the biologic solder is gradually absorbed during the normal wound healing process. Lauto A, Trickett R, Malik R, Dawes J M, Owen E R, “Laser-activated solid protein bands for peripheral nerve repair: an vivo study,” Lasers Surg Med. 1997;21(2):134-41 and Lauto A, Kerman I, Ohebshalon M, Felsen D, Poppas DP, “Two-layer film as a laser soldering biomaterial,” Lasers Surg Med. 1999;25(3):250-6.Biologic solders may be combined with wavelength specific chromphores. Talmor M, Bleustein C B, Poppas D P, “Laser tissue welding: a biotechnological advance for the future,” Arch Facial Plast Surg. 2001 July-September;3(3):207-13 and Oz MC, Johnson J P, Parangi S, Chuck RS, Marboe C C, Bass L S, Nowygrod R, Treat M R, “Tissue soldering by use of indocyanine green dye-enhanced fibrinogen with the near infrared diode laser,” J Vasc Surg. 1990 May;11(5):718-25. This provides both increased target specific energy absorption and decreased thermal energy leakage to surrounding tissue. In addition, by choosing a particular chromophore such as carbon black, fluorescein dye or indocyanine dye, an objective basis can be provided for gauging the adequacy of the laser welding by providing a predictable color change which correlates with the adequacy ofthe laser weld.

The most widely studied LTW solder is comprised of albumin, hyaluronic acid, and indocyanine green dye as the chromophore. Multiple studies in a variety of tissues have demonstrated that this liquid solder is capable of producing durable welds utilizing laser energy which is both spatially and temporally specific to the solder. However, there are several drawbacks to this solder formulation. As a liquid, it is difficult to place in a non-dependent area without significant run-off. Additionally, it is easily diluted by blood or other fluids and therefore must be applied in a completely dry bed. Further, the solder lacks significant internal structural stability and cannot successfully seal across small gaps in tissue. Also, since the solder is employed in liquid faun, it does not have an internal structure capable of holding tissue together until afterthe laser energy is applied since water has to evaporate from this solder when lased to form the laser weld This may necessitate the use of other devices to hold tissue together prior to and during formation of the weld. Finally, axial shortening of the albumin matrix of this welding material tends to cause the welding material to shrink and pull on the adjacent tissue as the weld is formed which may compromise the weld under certain conditions.

Lauto, Antonio et al., “Chitosan Adhesive for Laser Tissue Repair: In Vitro Characterization,” Lasers in Surgery and Medicine, vol. 36, pages 193-201 (2005) (hereinafter “Lauto 2005”) discloses use of insoluble strips of a laser activated adhesive for laser tissue repair. The insoluble adhesive strips are synthesized from a gelatinous solution containing chitosan (2% w/v), ICG (0.02% w/v) and acetic acid (2% w/v) (See page 194). The gel material of Lauto 2005 is very viscous and brittle when dried and thus Lauto 200.5 prepares insoluble strips of the material for use in laser welding. The strips were laser welded to moistened sheep intestine and demonstrated a tensile strength of 14.7 kPa and elastic modulus of 6.8 MPa. Lauto 2005 also suggested that the adhesives may potentially be used to deliver therapeutic compounds. Lauto 2005, however, fails to disclose use of a cross-linking agent in the formulation.

Lauto, Antonio et al., “In Vitro and In Vivo Tissue Repair with Laser-Activated Chitosan Adhesive,” Lasers in Surgery and Medicine, vol. 39, pages 19-27 (2007) (hereinafter “Lauto 2007”) discloses two formulations form making insoluble strips of a laser activated adhesive for laser tissue repair: Formulation I: a gelatinous solution containing chitosan (1.8% w/v), ICG (0.02% w/v) and acetic acid (2% w/v); and Formulation II: a gelatinous solution containing chitosan (1.8% w/v), ICG (0.02% w/v), genipin cross-linking agent (1% w/v), acetic acid (2% w/v) and ethanol (0.7% w/v) Lauto 2007 does not indicate that the gelatinous solutions used to prepare the insoluble adhesive strips used in the laser tissue repair method are supersaturated gels. Lauto2007 also concluded that “intermolecular and intramolecular cross-linking impaired the binding capability of collagen and chitosan.”

Ono, K et al., “Photocrosslinkable Chitosan as a Biological Adhesive,” Journal of Biomedical Material Resources, 49, 289-295 (2000) (hereinafter “Ono”), teaches a gel containing chitosan which has been modified with lactobionic acid and p-azidebenzoic acid. The gel is intended for use to seal pin sized holes in the small intestine, aorta and trachea. The addition of the azide and lactose moieties to the chitosan produced a highly water soluble chitosan solution subject to fluid dilution. Upon UV irradiation, the modified chitosan crosslinks with itself to produce an insoluble chitosan hydrogel matrix.

International Publication No. 2007/082292 (hereinafter “McGurk”) discloses a biologic glue that may be synthesized from a cross-linking agent and a protein, such as albumin, and/or various additives that may be formulated as a gel or hydrogel having an adjustable viscosity. Among various options, McGurk suggests that the implantable hydrogels which may be used in the invention may include chitosan. The materials of McGurk may include a fluorescent dye but McGurk does not appear to contemplate use of a laser specific chromophore. Additionally, the gel if McGurk may create a water tight seal, may be applied to wet tissue and may be used for various applications including filling voids, repairing tissue lacerations and tissue dissection.

International Publication No. 2008/053432 (hereinafter “Pini”) discloses a number of solid and semi-solid ICG formulations for use in various laser tissue welding applications, including cornea and skin repair. In one embodiment, the composition includes a supersaturated ICG gel that may be inserted and coated around a corneal incision using a front chamber cannula and laser welded. Excess gel may be washed away from the incision. In one embodiment, Pini discloses a semi-solid composition that may selectively include chitosan (0.5-15% w/w), ICG (0.5 -10% w/w) and any other substance that stabilizes the formulation. Pini, however, does not disclose a cross-linkable compound such as albumin in combination with chitosan and ICG nor does Pini appear to contemplate cross-linking in its composition.

U.S. Pat. No. 5,958,443 (hereinafter “Viegas”) discloses a gel composition including a film forming polymer, an ionic polysaccharide and a counter-ion that may be used as a drug delivery system, a laser ablatable shield or a corneal protective composition. The viscosity of the gel composition may be adjusted. Viegas discloses that the gel may selectively include chitosan and a plurality of cross-linkable compounds such as polyvinyl alcohol and hyaluronic acid if an irreversible gel or gel that retains its shape is required. Viegas fails to disclose use of a laser specific chromophore or a method for laser tissue welding.

WO 92/14513 (hereinafter, “Sawyer”) discloses use of a filler material for laser tissue welding. Sawyer employs a solid collagen “filler” to effect the weld as a solid rod, flake, etc. with the cited advantage of reducing shrinkage of the wound which may occur during welding resulting in contracting of the solder away from the wound edges. Sawyer notes that fillers gels such as gelatin are rapidly dissolved by blood as they are highly soluble.

Accordingly, there remains a need in the art for a solder formulation for use in laser tissue welding that has sufficient viscosity to be applied in a variety of specialized applications while at the same time providing sufficient bond strength to create a reliable tissue weld.

### SUMMARY OF THE INVENTION

In a first aspect, the invention relates to supersaturated gel formulations. The supersaturated gel composition includes a solution of chitosan, albumin, and a laser specific chromophore.

In a second aspect, the invention relates to laser tissue welding methods using the gel formulations of the present invention. In the methods, the gel formulation of the present invention is provided to a site for tissue repair and the laser specific chromophore within the gel is excited with a laser in order to fuse tissue by inducing protein denaturation.

In a third aspect, the gel formulations and laser tissue welding methods may be used, for example, to enable skull base repairs, aerodigestive endoscopic repairs, endoscopic endonasal surgical repairs, iatrogenic esophageal perforation repairs, laparoscopic abdominal surgical repairs, lung repairs, colon repairs, anastomosis of vessels, urologic/gynecologic endoscopic pelvic repairs, orofacial surgical repairs, dental replacement, skin closure, uterine closure and repairs after fibroidectomies and bladder surgery.

In a fourth aspect, the present invention relates to a method for preparing supersaturated gel compositions for use in laser tissue welding. In this method, an acidic aqueous solution of chitosan is combined with an aqueous albumin solution. Subsequently, an aqueous indocyanine green dye solution is added. The resultant solution is allowed to precipitate and the supernatant is removed A supersaturated gel composition is obtained.

### BRIEF DESCRIPTION OF THE DRAWINGS

FIG. 1A shows scanning electron micrographs of the would healing at 0 post-operative days after application of laser tissue welds as in Example 3 (on the right) and Comparative Example C (on the left).

FIG. 1B shows scanning electron micrographs of the wound healing at 5 post-operative days after application of laser tissue welds as in Example 3 (on the right) and Comparative Example C (on the left).

FIG. 1C shows scanning electron micrographs of the would healing at 15 post-operative days after application of laser tissue welds as in Example 3 (on the right) and Comparative Example C (on the left). This image demonstrates that the presence of the solder acts as a scaffold for normal wound healing and scarring to occur and does not impair remucosalization of the underlying maxillary sinus.

FIG. 2 shows a scanning electron micrograph of the gel solder of Example 3 prior to laser welding on the left and the same gel solder of Example 3 after laser welding on the right.

FIG. 3 shows the return of baseline nerve function as measured by electromyography as measured in Example 7 and Comparative Example F.

FIG. 4 shows the operative time by method of repair for the nerve repair procedure of Example 7.

FIG. 5 shows the learning curve for repair time based on the number of procedures carried out.

FIG. 6 shows the mean rabbit tympanic membrane failure pressure for a control versus a laser weld of the present invention with an underlay graft.

### DETAILED DESCRIPTION OF THE PREFERRED EMBODIMENTS

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

It must be noted that as used herein and in the appended claims, the singular forms “a”, “an”, and “the” include plural references unless the context clearly dictates otherwise. As well, the terms “a” (or “an”), “one or more” and “at least one” can be used interchangeably herein. It is also to be noted that the terms “comprising”, “including”, and “having” can be used interchangeably.

Laser tissue welding (LTW) involves the application of a protein based solder doped with a laser specific chromophore which fuses tissue edges through extracellular matrix protein denaturation following laser exposure. These welds can be created endoscopically utilizing a flexible fiber optic cable and have been shown to have useful bond strengths. Additionally, the protein based solder has been demonstrated to provide a scaffold for normal wound healing progression while obviating the need for foreign body implantation or additional surgery for removal.

In a first aspect, the invention relates to supersaturated gel formulations useful, for example, in laser tissue welding. The supersaturated gel composition includes a solution of chitosan, albumin, and a laser specific chromophore.

The most preferred chromophore for use in the present invention is indocyanine green (ICG). However, any other type of biocompatible dye with suitable chemical and physical features and properties for use in laser welding applications may be used as an alternative including but not limited to carbon black and fluorescein dye. IC-GREEN pharmaceutical form, produced by Akorn, Buffalo, Ill., USA and ICG-PULSION, PULSION Medical System AG, Monaco (Germany) are examples of ICG chromophores. An aqueous solution of ICG has an optical absorption spectrum characterized by two peaks around 700 and 780 nm respectively. The relative intensity of these peaks changes with the concentration of the solution and/or different degrees of super-saturation.

In the present invention a supersaturated gel formulation is employed. As a supersaturated gel formulation, the composition of the present invention does not suffer from the drawbacks encountered by traditional liquid solders which include difficulty of placement in non-dependent areas, rapid dilution by blood or other fluids, inability to seal across small gaps in tissue due to a lack of significant internal structural stability in the conventional solder formulations and the need to evaporate significant quantities of water from the solutions during the laser welding process.

The supersaturated gel formulation of the present invention preferably exhibits rheological behavior which allows placement at a desired welding location without significant runoff of the formulation. More preferably, the supersaturated gel formulations of the invention have a viscosity (measured in Saybolt Seconds Universal (SSU) at 16° C.) of from about 700 to about 250,000, even more preferably the viscosity of the supersaturated gel formulations is from about 2500 to about 70,000, and, most preferably, the viscosity of the supersaturated gel formulations is from about 7000 to about 25,000.

As measured prior to precipitation and supersaturated gel formation and based on the dry weight of the chitosan, ICG and albumin components, the formulations of the present invention may contain from about 0.5-7.0% (w/w) chitosan, about 0.05 to about 0.60% (w/w) ICG and about 20-99% (w/w) albumin, the balance being solvent/carrier material. A preferred formulation may contain from about 0.7-5.5% (w/w) chitosan, about 0.07-0.55% (w/w) ICG; and about 24-99% (w/w) albumin, with the balance being solvent/carrier material. More preferred formulations may contain from about 2.3-4.0% (w/w) chitosan; about 0.2-0.36% (w/w) ICG; and about 72-97.5% (w/w) albumin, with the balance being solvent/carrier material. Most preferably, the formulations may contain about 2.9-4.0% (w/w) chitosan; about 0.2-0.3% (w/w) ICG; and about 91-96.9% (w/w) albumin, with the balance being solvent/carrier material. A particularly preferred formulation contains, as measured prior to precipitation and supersaturated gel formation, about 3.1% (w/w) chitosan; about 0.3% (w/w) ICG; and about 96% (w/w) albumin, with the balance being solvent/carrier material.

When alternative chromophores other than ICG are employed, the skilled person can determine the amount of chromophore to employ in the compositions of the present invention based on factors such as the viscosity of the gel composition and the desired level of laser light absorption.

Suitable solvent/carrier materials include solvents capable of dissolution of the various ingredients of the composition. The solvent/carrier materials should preferably be biocompatible and water is a particularly good solvent material for the formulations of the present invention. Other suitable solvents known to skilled persons may also be employed in the invention, as well as mixtures thereof.

Chitosan refers to an amino-polysaccharide derived from the deacetylation of chitin which is found in crustacean shells and can be engineered to form a cationic polymer. The chitosan component of the formulation can best be dissolved in a suitable solvent under acidic conditions. Thus, it is typically desirable to employ a sufficient amount of a suitable biocompatible acid component in the formulations of the invention to facilitate chitosan dissolution. A particularly preferred acid for use in the formulations of the present invention is acetic acid, though other suitable acids known to skilled persons may also be employed, as well as mixtures thereof.

The chitosan component of the present invention offers several advantages. For example, it has been found that use of the chitosan component provides very good bond strengths and/or burst pressures in laser welding applications. In addition, chitosan is hemostatic, mucoadherent and biodegradable making it well suited for use in laser welding and tissue repair applications.

The albumin component is provided for the purpose of at least partially cross-linking the chitosan component as well as improving the binding strength of the chitosan component to tissue. In this manner, the desired rheological behavior of the formulations can be achieved. More preferably, sufficient albumin is employed to substantially completely crosslink the chitosan component of the formulation.

It has been found that the formulations of the present invention provide rheological properties which facilitate delivery to the site of laser welding, as well as maintenance of the formulation in place during the laser welding procedure. Also, the formulations of the present invention provide sufficient chemical stability to allow the formulations to be prepared in advance of use and stored under suitable storage conditions. Suitable storage conditions may, for example, involve refrigeration at about 4° C. in the absence of light in order to protect the chromophore.

Other suitable additives may be employed in the formulation such as antioxidants, antibacterial agents, steroids, antifungals, antivirals, fibroblast inhibitors, antibiofilm agents, anti-inflammatories, immunologically active compounds, isotonizing agents, pH modulating agents, plasticizers, nanoparticles, and antibiotics. Sufficient amounts of each of these agents may be employed to accomplish the desired function in the formulation of the present invention. The supersaturated gels of the invention are capable of reversibly binding pharmaceutical agents which will elute over time in vivo Thus unlike traditional formulations, the solder could also be used as a drug delivery vehicle for a variety of thermally stable compounds.

One suitable method for preparation of a supersaturated gel formulation in accordance with the present invention is as follows. An acidic aqueous solution of chitosan is stirred and to this may be added an aqueous albumin solution. Subsequently, an aqueous indocyanine green dye solution is added. The solution is allowed to precipitate and the supernatant is removed and a supersaturated gel composition is obtained. The weight percentage ranges in this application for chitosan, albumin and ICG contents are determined based on the dry weight of the chitosan, albumin and ICG components prior to this precipitation step and gel formation. Further steps may be taken to remove the supernatant, as needed.

In some embodiments of the invention, the order of addition of the ingredients may be important to determining the properties of the final supersaturated gel product and/or the laser weld formed therefrom. In such embodiments, it is preferred to add the albumin solution to the chitosan solution prior to addition of dye and subsequent precipitation of the supersaturated gel. In this manner, a concentrated form of the albumin may be prepared which does not shrink as much as some other laser solders during laser welding.

The method of the present invention provides a supersaturated gel which is more viscous than a liquid solution but is sufficiently pliable that it can be pumped to the location of the laser weld and can be formed into a desired shape which can be retained through the laser welding step. Further, since the supersaturated gel of the present invention is precipitated from aqueous solution, it has the additional benefit that it is water insoluble and thus it is not necessary to apply the gel to a dry tissue bed for the laser welding process. This greatly increases the flexibility of the laser welding process while at the same time eliminating the need to take additional preparatory steps to provide a dry tissue bed for some welding scenarios.

Further, the supersaturated gel of the present invention exhibits hemostatic properties when applied. Also, the gel can be delivered with a pharmaceutical carrier material, if desired.

The supersaturated soldering gel of the present invention has been found to be stronger than currently described solders, is easier to manipulate and precisely place, is able to bridge small tissue gaps of up to several millimeters across, and can be used as a carrier for pharmaceutical agents. In addition, since the material of the present invention is a gel, it has sufficient structure to hold some tissue together during the laser welding process, thereby reducing the need for external means to hold tissue in place and/or closely opposed the tissue edges for welding. This facilitates, for example, endoscopic laser tissue welding where it may be difficult to hold tissue in place with other tools during the welding process.

Another advantage of the gel of the present invention is that it does not shrink as much as comparable laser welding solders such as those disclosed in International application publication no. WO 92/014513. It is believed that the precipitation method of the present invention concentrates the albumin in the supersaturated gel in a manner which reduces the shrinkage of the material during laser welding.

A further advantage of the gel of the present invention is that evidence shows that the gel interpolates into tissue prior to or during the laser welding process thereby forming a more structurally sound bond. The solid insoluble strips of Lauto 2005, for example, do not appear to be able to interpolate into the tissue and thus lack this advantageous feature of the invention.

The chitosan/albumin supersaturated soldering gel of the present invention is capable of producing water tight tissue bonds which exceed intracranial pressure, support native wound healing, and produce negligible collateral thermal tissue injury. These welds can be produced using laser light provided via a fiber optic cable and thus are potentially ideally suited for endoscopic application. This soldering gel is stronger and easier to use than traditional solders and can be used as a carrier for pharmaceutical agents. While this technology is well suited for skull base repair, its utility extends into any surgical specialty where water tight tissue closure is required in an area with difficult access including but not limited to head and neck surgical repairs such as skull base repairs, tracheal repairs, aerodigestive endoscopic repairs, endoscopic endonasal surgical repairs, iatrogenic esophageal perforation repairs, laparoscopic surgical repairs such as laparoscopic abdominal surgical repairs, lung repairs, colon repairs, repair of the tympanic membrane (ear drum), neurological repairs such as reattachment of severed nerves, anastomosis of vessels, urologic/gynecologic endoscopic pelvic repairs, orofacial surgical repairs, dental replacement, skin closure, thoracic surgical repairs, neurosurgery repairs, uterine closure and repairs after fibroidectomies and bladder surgery. The method can be used for repairs in, for example, general surgery, natural orifice transluminal endoscopic surgery (NOTES), transoral gastroplasty (TOGA), laparoscopic surgery, video-assisted surgery such as video-assisted thoracic surgery (VATS) and/or robotic surgery.

The invention is particularly suitable for applications where suturing or stapling is not feasible, e.g. where a certain degree of water or airtightness may berequired of the repair. For example, in thoracic surgery, a certain minimum airtightness may be required of the repair which can be achieved using the material of the present invention. Similar concerns may apply in various forms of head and neck surgery where fluid leakage must be minimized.

In a second aspect, the invention relates to laser tissue welding methods using the gel formulations of the present invention. In the methods, the gel formulation of the present invention is provided to a site for tissue repair and the laser specific chromophore within the gel is excited with a laser in order to fuse tissue by inducing protein denaturation.

For the purposes of the present invention and according to one embodiment thereof, a super-saturated gel formulation of the present invention is delivered to the site of tissue repair. Laser tissue welding may be performed, for example, using laser light having a wavelength of from about 650-850 nm. This may be accomplished, for example, using an AlGaAs diode laser with emission at 810 nm. Alternatively, a diode laser module (Iridex, Mountain View, Calif.) may be utilized coupled to a 600 μm core diameter quartz silica fiber optic cable with the following specifications: power: 1.0 W, pulse duration: 0.5 s, pulse interval: 0.1 s, power density: 31.81 d/cm², major wavelength output: 808 +/−1 nm. Sufficient laser emission is employed to create a suitable tissue bond by protein denaturation. For example, during welding, laser energy may applied to the solder until a characteristic green to beige color transition occurs or until a specific temperature is reached. Skilled persons can routinely determine a sufficient degree of lasing for the purposes of performing a particular tissue repair operation based on factors such as the size and location of the tissue repair as well as the amount of solder required to effectively seal the defect.

The burst pressure results of the examples below demonstrated several important characteristics. An immediate burst pressure of 135.03 +/−5.76 mmHg which rose to 154.10 +/−3.68 mmHg by post-op day 5 was achieved in Example 1 without the need for an additional periosteal graft which confirms that laser welding in sinonasal mucosa can produce a weld with burst strengths exceeding even pathologically elevated intracranial pressure. A significant improvement relative to the similar Comparative Example A using a conventional formulation based on a mixture of hyaluronic acid, albumin and ICG was also demonstrated. Also the examples verified that the purpose of the weld to act as a scaffold to reinforce the repair and prevent CSF leakage until native scar formation can occur was also achieved. Example 2 also showed a significant improvement relative to the similar Comparative Example B using a conventional formulation based on a mixture of hyaluronic acid, albumin and ICG. The remaining examples show that the present invention can be successfully applied to a variety of other types of repairs.

### EXAMPLES

Materials and Methods

Burst Threshold Manometry: The manometry system is comprised of a closed saline filled system with a traceable manometer (range −776.00 to +776.00 mmHg, Fisher Scientific, Pittsburgh, Pa.) and a 10 cc syringe arranged in parallel utilizing standard intravenous tubing secured by luer lock. To measure the burst pressure of the mucosal repair, the rabbit is sacrificed and the mucosal repair is exposed following removal of the silastic spacer. An additional sinusotomy is created with a 1 mm otologic diamond burr on the anterolateral aspect of the maxillary sinus. A luer lock is then bonded over the sinusotomy using dental cement (Stoelting Co., Wood Dale, Ill.) in a water tight fashion and connected in parallel to the manometry system. The native maxillary ostium is identified within the nasal cavity and occluded with a strip of mucosa which is then reinforced with dental cement. The pressure in the system is incrementally increased by depressing the plunger on the syringe and burst pressure is recorded at the point where saline ruptures through mucosa. This is then correlated to the maximal pressure recorded on the manometer.

Histologic Analysis: Following burst pressure analysis, a single repair is chosen from each condition and harvested along with the surrounding bone and imbedded in paraffin. Standard hematoxylin and eosin staining is performed and repairs are graded at two distinct cuts on a 3 point scale by a blinded veterinary histopathologist for collateral thermal injury, degree of local inflammation, and fibroplasia.

Statistical Analysis: All statistical analyses are performed using SigmaStat v3.1 (Systat Software Inc, San Jose, Calif.). The burst pressure data is ranked and a 2-way analysis of variance (ANOVA) is performed with factors of condition (laser weld vs. open) and post-operative day (0, 5, or 15). Post-hoc pairwise multiple-comparisons are made using the Tukey Test with a significance level set at a probability of 0.05.

Laser System: A diode laser module (Iridex, Mountain View, Calif.) is utilized coupled to a 600 μm core diameter quartz silica fiberoptic cable with the following specifications: Power: 0.5-1.0 W, Pulse Duration: 0.5 s, Pulse Interval: 0.1 s, Power Density: 31.81 d/cm² to about 19 W/cm², Fluency 8.0 J/cm², Major Wavelength Output: 808 +/−1 nm.

Sample Size: The formula below is used to calculate the sample size for the respective comparisons in this study. This formula is used considering alpha error with za as specified.

$$N = \frac{{( {z}_{a} )}^{2} \times 2 \times s 2}{d 2}$$

(z=value for alpha error [1.96]; s2 =variance; d=difference to be detected; N=number of subjects per study group). A minimum of 4 subjects per study group are utilized since our sample size calculation demonstrated a need for>3 subjects for each study group for adequate power.

### Example 1

Production of Chitosan/Albumin Solder

Stock solutions:

1) 0.2 M acetic acid—12.01 g glacial acetic acid+H₂O to total of 1 L

2) 1.3% (w/w) chitosan—Mix 0.327 g of 88-92% DDA Chitosan (Ultrasan CHO2) in 12 mL H₂O and 12 mL 0.2 M acetic acid over 36 hours on a rocker.

3) 71.4% (w/w)—indocyanine green dye (Cardiogreen Sigma). Combine ICG with H₂O for 2.5 mg/mL solution. Protect from light with foil.

4) 29.4% (w/w) albumin solution—Mix 2.5 g into 6 mL albumin. Heat in a 37° C. water bath for 15 minutes and vortex as needed. Centrifuge at 3000 rpm for 3 minutes to remove bubbles.

Solder Production:

1) Add 4 cc of the 1.3% chitosan stock solution to a 50 cc beaker over a stir bar,

2) Add 4 cc of the 29.4% albumin stock solution to the chitsoan solution while stirring, and

3) Add 2 cc of the indocyanine green dye solution to the mixture.

4) The solution then precipitates and the supernatant is removed. The gel is aspirated into a 10 cc syringe and, optionally the mixture may be vortexed to remove additional supernatant, if needed.

The final product is a supersaturated gel formulation in accordance with the present invention. The supersaturated gel formulation was then used to evaluate laser weld burst strength (the pressure at which the weld ruptured) in an in vivo surgically created rabbit maxillary sinusotomy. The traditional solder was able to achieve an immediate burst strength of 120.85 +/−47.84 mmHg and rose to 132.56 +/−24.02 mmHg by post-op day 5 group, as shown in Comparative Example A below. Of note with the traditional albumin/hyaluronic acid solder, a periosteal graft was required to bridge the gap in the sinusotomy prior to welding. When the experiment of Comparative Example A was repeated with the present supersaturated gel formulation as the solder, the immediate burst strength achieved was 135.03 +/−5.76 mmHg which rose to 154.10 +/−3.68 mmHg by post-op day 5, without the need for an additional periosteal graft. In both groups histologic analysis demonstrated normal wound healing, negligible collateral thermal injury and minimal inflammatory responses.

### Example 2

This example used the supersaturated gel formulation of Example 1 to evaluate weld burst strength in an explanted rabbit esophagotomy model. A full thickness perforation was created in a rabbit esophagus and following welding, the burst pressure was measured. Using the traditional solder, a burst strength of 71.6 +/−7.5 mmHg was achieved, as demonstrated below in Comparative Example B. However this required tacking sutures to keep the wound from deforming during burst pressure measurement. When the experiment of Comparative Example B was repeated with the present supersaturated gel formulation as the solder, a burst strength of 95.86 +/−8.9 mmHg was achieved and no tacking sutures were required.

The histologic analysis of the repairs was also quite favorable. No difference was found between overall degree of inflammation and fibroplasia between the laserweld and control groups. This again supports the fact that scar formation may progress unimpeded despite the persistence of solder on post-operative day 15.

Of equal importance was the lack of thermal injury to the surrounding tissue. The addition of a laser wavelength specific chromophore results in enhanced efficiency in solder energy absorption thereby allowing effective welding with a relatively low laser energy density. This is of importance when assessing the potential utility of this technology for use within close range of sensitive body parts such as the anterior cranial fossa and its associated structures.

### Comparative Example A

Solder Preparation: The preparation of the biologic solder of this comparative example is based on previously described techniques found, for example, in Kirsch, A. J., Miller, M. I., Hensle, T. W., et al., “Laser tissue soldering in urinary tract reconstruction: first human experience,” Urology 1995; 46(2):261-6. The solder is comprised of a 2:1:2 mixture of 42% bovine serum albumin (Fisher Scientific, Pittsburgh, Pa.), indocyanine green dye (2.5 mg/mL, Sigma-Aldrich, St Louis, Mo.), and hyaluronic acid sodium(10 mg/mL, Sigma-Aldrich, St Louis, Mo.), respectively. The albumin solution is filtered through a 0.2 μm pore filter and 4004 aliquots are mixed with 200 μL of indocyanine green dye and 4004, of hyaluronic acid.

Bilateral maxillary sinus mucosal incisions were made in twenty New Zealand White Rabbits and one side was repaired using LTW. Burst pressure thresholds were measured on post-operative day 0, 5, and 15 and were compared to control using a 2-way ANOVA and a post-hoc Tukey test. Welds were examined histologically for thermal injury, inflammation, and fibroplasia and graded on a 3-point scale by a veterinary histopathologist.

Results: The burst pressures of the LTW group were significantly higher than control on post-operative day 0 (120.85 mmHg, N⁼4, SD=47.84 vs. 7.85 mmHg, N=4, SD=0.78), and day 5 (132.56 mmHg, N⁼8, SD=24.02 vs. 41.7 mmHg, N⁼8, SD=7.2)(p<0.05). By post-operative day 15 there was no significant difference between LTW (169.64 mmHg, N=8, SD=18.49) and control (160.84 mmHg, N=8, SD=14.16) burst thresholds. There was no evidence of thermal injury to the surrounding tissue in any group as well as no difference between experimental group and control with respect to inflammation or fibroplasia.

### Comparative Example B

Iatrogenic esophageal perforation is a potentially morbid complication whose incidence has risen over the past two decades secondary to increased rats of diagnostic and therapeutic esophageal endoscopy. The present example utilizes animal model for primary, single stage, transluminal repair of esophageal perforation utilizing laser tissue welding technology which provides an immediate, water tight closure without the need for foreign body implantation.

Iatrogenic injury during esophageal instrumentation accounts for as much as 59% of all esophageal perforations and occurs in 0.03% of flexible and 0.11% of rigid esophagoscopy. Mortality rates have been reported at 4-20% when treatment is initiated within 24 hours and can double with a delay beyond 48 hours. These injuries tend to occur at anatomic narrow points including the cricopharyngeus, aortic arch, left mainstem bronchus, and lower esophageal sphincter.

Solder Preparation: The preparation of the biologic solder is carried out as in Comparative Example A.

Rabbit Tissue Harvest: Twenty New Zealand White rabbits utilized were sacrificed under an unrelated institutional IACUC protocol and approval was obtained for use of post-mortem tissues. A midline incision was made from sternal notch to pubis followed by a median sternotomy to expose the thoracic esophagus. The tracheoesophageal complex was dissected off the prevertebral fascia and truncated superiorly at the cricopharyngeus and inferiorly at the level of the diaphragm. The esophagus was then dissected off the trachea in its entirety.

Experimental Groups: Our study consisted of testing the burst pressure through an esophageal injury under four conditions including 5 mm open incision, external suture closure using 2 5-0 interrupted prolene stitches, external laser augmented suture closure, and sutureless endoluminal laser weld. All conditions were tested 5 times.

Histology: Five additional endoluminal welds were harvested and imbedded in paraffin. Standard hematoxylin and eosin staining was performed and welds were examined by a veterinary histopathologist for collateral thermal tissue injury.

The maximal pressure achievable in the closed manometry system was 186.4 mmHg The average burst threshold was 6.5 mmHg (N=5, SD=1.94) in the open incision group and 37.18 mmHg (N=5, SD=1.97) in the external suture group. Among the laser welding conditions, the external laser augmented suture group achieved an average burst strength of 71.60 mmHg (N=5, SD=7.58) while the endoluminal group demonstrated an average burst strength of 54.78 mmHg (N=5, SD =5.84).

The differences in the median values among the treatment groups were all significantly greater than would be expected by chance (Kruskal-Wallis, H=17.87, 3 df, P=<0.001). Post hoc analysis indicated several treatment groups had significantly different burst strengths. The burst strength of the endoluminal welding group was significantly higher than that of the open incision group (P<0.05). The burst strength of the external laser augmented suture group was significantly higher than both the open incision and the external suture alone group (P<0.05). There was no statistically significant difference between the endoluminal weld group and the external suture or external laser augmented suture group.

Histologic examination of lased coagulum was compared to normal mucosal controls and demonstrated negligible thermal tissue injury.

### Example 3 and Comparative Example C

In Example 3 a supersaturated gel solder containing 1.3% by weight of chitosan, 29.4% albumin and indocyanine dye prepared in accordance with the present invention was employed for skull base tissue welding. In Comparative Example C, a prior art solder comprising 42% albumin solution, indocyanine dye and hyaluronic acid sodium was employed for skull base tissue welding after an operation. The laser system described above was used in these examples.

Burst threshold manometry was employed to evaluate the burst strength of the welds. The immediate burst pressure (post-operative day 0) of the control without a tissue weld was 7.85 mmHg, the weld of Comparative Example C had an immediate burst pressure of 120.85 mmHg and the weld of Example 3 of the present invention had an immediate burst pressure of 135.02 mmHg On post-operative day 5, the control had a burst pressure of 41.7 mmHg, the weld of Comparative Example C had a burst pressure of 132.56 and the weld of Example 3 of the present invention had a burst pressure of 1.54.10 mmHg.

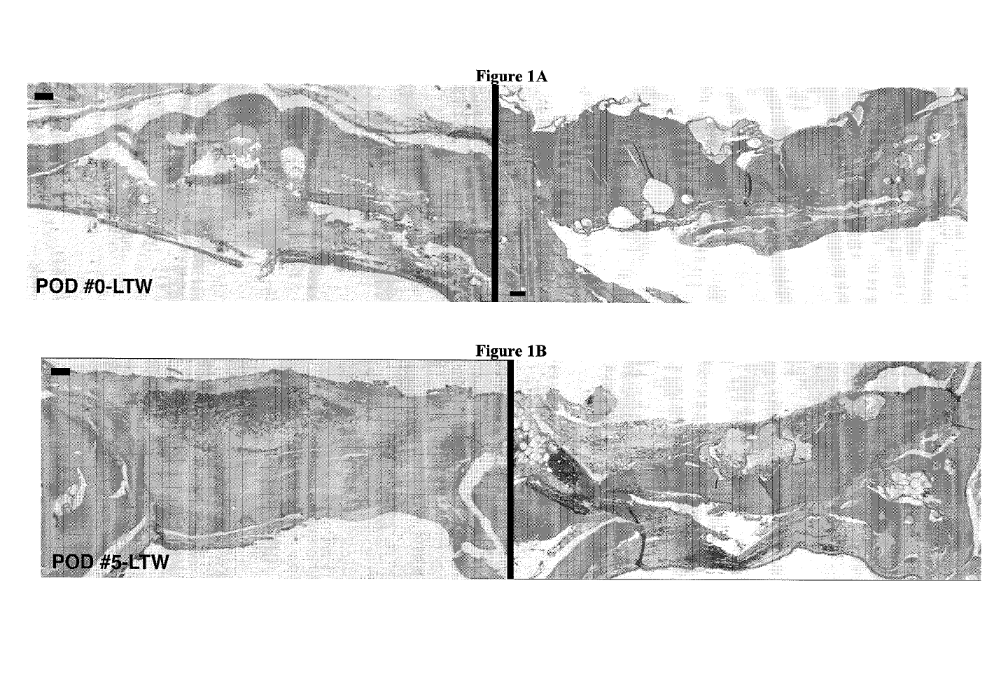

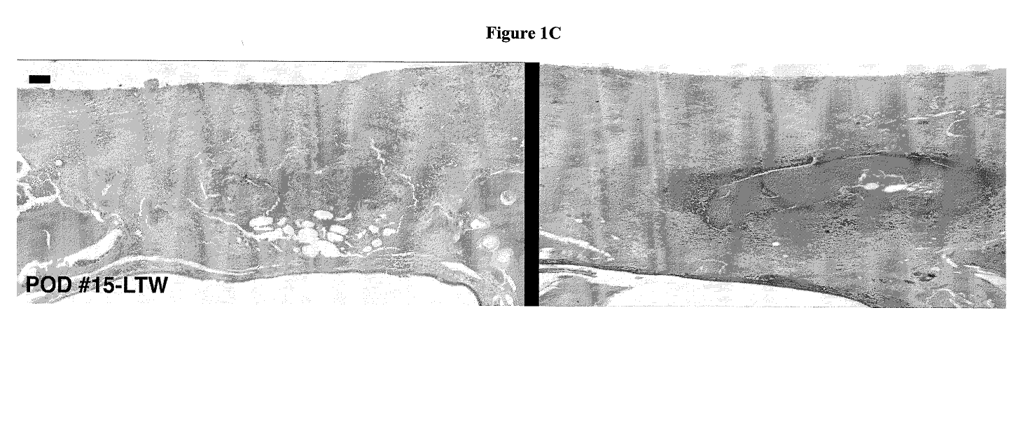

FIGS. 1A-1C show the wound healing of Comparative Example C (on the left) and Example 3 (on the right) at 0 post-operative days, 5 post-operative days and 15 post-operative days. From these figures it can be seen that the application of the solder results in negligible collateral thermal injury and allows for the progression of normal wound healing processes. The solder is absorbed over time with minimal inflammation and is shown to be completely resorbed by day 45. Additionally this example demonstrates that the chitosan based solder does not require a bridging graft to successfully close the wound.

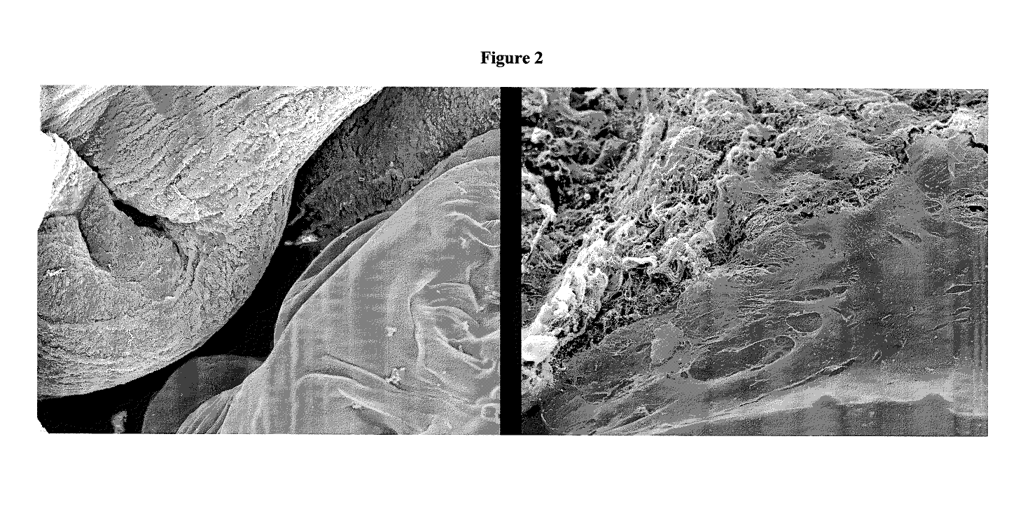

FIG. 2 shows scanning electron micrographs of the tissue repair of Example 3 prior to welding but after application of the solder (on the left) and after lasing of the same applied solder (on the right). This figure demonstrates that lasing of the solder of the invention results in inclusions of the lased solder within the tissue at the interface between the tissue and the lased solder.

### Example 4 and Comparative Example D

In these examples, the solders of Example 3 and Comparative Example C were employed in Example 4 and Comparative Example D, respectively, for esophageal repair. The laser system described above was used in these examples. The results obtained were as shown in Table 1 below.

TABLE 1

| Repair Mechanism | Burst Pressure (mmHg) |
|---|---|
| Open esophageal wound with no | 6.5 |
| repair mechanism. | |
| External Sutures only | 37.18 |
| Endoluminal laser tissue weld with | 54.78 |
| solder of Comparative Example D | |
| External Suture plus endoluminal | 71.60 |
| laser tissue weld with solder of | |
| Comparative Example D | |
| External laser tissue weld with the | 95.86 |
| solder of Example 4 | |

The results shown in Table 1 demonstrate that the laser tissue weld of the present invention had a significantly greater burst strength than the comparative laser tissue weld, even when the comparative laser tissue weld was combined with sutures.

### Example 5

In this Example, the solder composition of Example 3 was employed for tracheal repair. The laser system described above was used in this example. Three different types of tracheal repair were undertaken and the results are shown in Table 2 below.

TABLE 2

| | Burst Strength of | |
|---|---|---|
| | Injury With no Weld | Burst Strength of |
| Type of Repair | (mmHg) | Weld (mmHg) |
| Membranous | 1.72 | 101.00 |
| Injury/Weld | | |
| Excised Cartilage | 1.64 | 77.34 |
| Injury/Weld | | |
| Replaced Cartilage | 1.68 | 75.08 |
| Injury/Weld | | |

The results given in Table 2 show that the laser tissue welding materials and methods of the present invention are applicable for a variety of types of tracheal repair.

### Example 6 and Comparative Example E

In this example, the solder formulation of Example 3 was used for laser tissue welding of lung tissue. The burst strength of the weld was compared to a baseline value of the burst strength of healthy lung tissue, to a repair done with Tisseel fibrin sealant and to unrepaired tissue. The results are given in Table 3.

TABLE 3

| | Burst Strength | |
|---|---|---|
| Type of Repair | (mmHg) | Percent of Baseline |
| Healthy tissue no | 22.5 | 100 |
| repair | | |
| Laser Tissue Weld | 20 | 89 |
| Tisseel | 10.7 | 48 |
| Unrepaired | 8 | 35 |

These results show that the laser tissue weld provided significantly greater burst strength than other methods of lung tissue repair. The burst strength was measured immediately after creating an iatrogenic perforation or after the repair was performed.

### Example 7 and Comparative Example F

In this example, repair of a severed rabbit facial nerve was undertaken. Three were repaired using conventional suture an astomosis with three 9-0 monofilament nylon sutures on an atraumatic taper needle and three were repaired by laser welding in using the solder composition of Example 3 in accordance with the present invention.

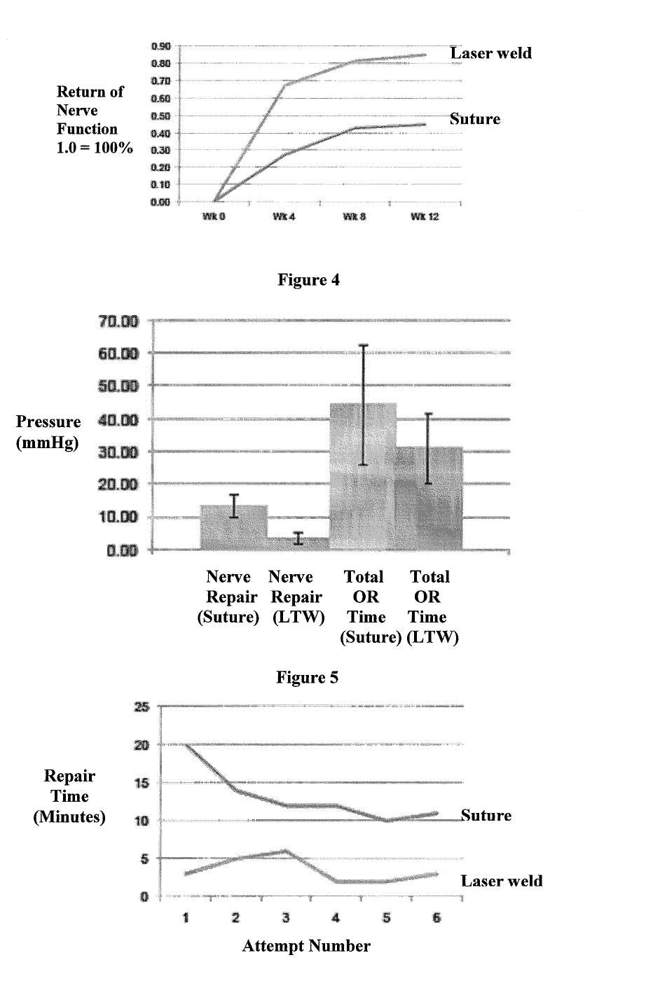

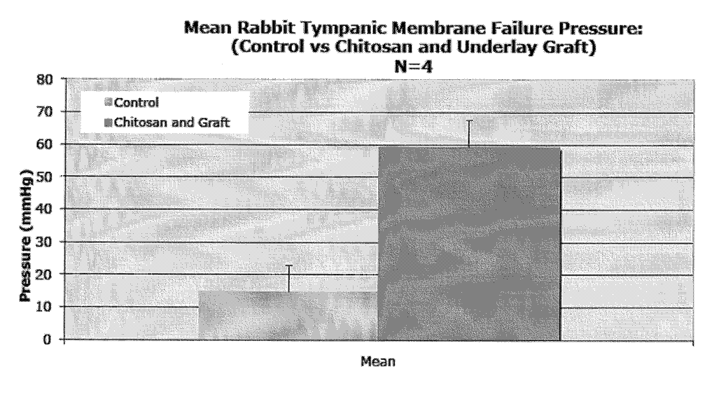

Surgical time was assessed and the rate/degree of nerve function recovery over 12 weeks was measured by electromyography. FIG. 3 shows the return of baseline function as measured by electromyography. FIG. 4 shows the operative time by method of repair for the nerve repair procedure of Example 7. FIG. 5 shows the learning curve for repair time based on the number of procedures carried out. The results show that laser tissue welding repair of rabbit facial nerve resulted in a greater return of function over 12 weeks than traditional suture repair as measured by electromyography. Also, operative procedures carried out using laser tissue welding repair were up to five times faster than traditional suture repair. Finally, the repair time using laser welding is practically independent of the surgeon's experience and thus can be performed by a novice nearly as fast as by a surgeon experience with the procedure, whereas there is a significant learning curve for suture repair.

### Example 8

The middle ear of a rabbit was insufflated and the pressure at which the ear drum ruptured was measured. In order to prepare these specimens, the entire pars flaccida of the tympanic membrane was treated with a combination of the suction and a fine otologic pick. Periosteum of the temporal bone was harvested and pressed into a thin fascial graft, which was then placed in an underlay fashion through the tympanic membrane perforation. A thin layer of solder was next placed over the graft, paying close attention to overlap the junction of the fascial graft and the edge of the perforation The ear drum was then repaired using laser tissue welding with the solder formulation of Example 3 and the pressure at which the repair ruptured was measured following insufflation. The results are shown in FIG. 6. From these results, it can be seen that the laser tissue weld was up to five times stronger than the native uninjured ear drum tissue.

The foregoing examples have been presented for the purpose of illustration and description and are not to be construed as limiting the scope of the invention in any way. The scope of the invention is to be determined from the claims appended hereto.

What is claimed is:

1. A supersaturated gel composition suitable for use in laser welding of tissue comprising: chitosan, a laser specific chromophore and albumin

2. The composition of claim 1, wherein the composition comprises a sufficient amount of albumin to crosslink the chitosan.

3. The composition of claim 1, wherein the chitosan comprises from about 0.7-5.5% (w/w), based on the total weight of the composition as measured prior to precipitation and gel formation.

4. The composition of claim 3, wherein the albumin comprises from about 24-99% (w/w), based on the total weight of the gel composition as measured prior to precipitation and gel formation.

5. The composition of claim 1, wherein the chitosan comprises from about 2.3-4.0% (w/w), based on the total weight of the composition as measured prior to precipitation and gel formation.

6. The composition of claim 5, wherein the albumin comprises from about 72-97.5% (w/w), based on the total weight of the gel composition as measured prior to precipitation and gel formation.

7. The composition of claim 1, wherein the chitosan comprises from about 2.9-4.0% (w/w), based on the total weight of the composition as measured prior to precipitation and gel formation.

8. The composition of claim 7, wherein the albumin comprises from about 91-96.9% (w/w), based on the total weight of the gel composition as measured prior to precipitation and gel formation.

9. The composition of claim 1, wherein the gel composition has a viscosity of from about 700 to about 250,000 Saybolt Seconds Universal at 16° C.

10. The composition of claim 1, wherein the gel composition has a viscosity of from about 2500 to about 70,000 Saybolt Seconds Universal at 16° C.

11. The composition of claim 1, wherein the gel composition has a viscosity of from about 7000 to about 25,000 Saybolt Seconds Universal at 16° C.

12. A tissue repair method comprising the steps of:
providing a supersaturated gel composition comprising chitosan, a laser specific chromophore and albumin;
applying said gel composition to a tissue repair site; and
contacting said gel composition with laser light to produce a laser weld,
wherein at least a portion of said chitosan in said gel composition is cross-linked by said albumin.

13. A method as claimed in claim 12, wherein laser light having a wavelength of from about 650-850 nm is employed.

14. The method of claim 12, wherein the chitosan comprises from about 0.7-5.5% (w/w) and the albumin comprises from about 24-99% (w/w), based on the total weight of the gel composition as measured prior to precipitation and gel formation.

15. The method of claim 12, wherein the chitosan comprises from about 2.3-4.0% (w/w) and the albumin comprises from about 72-97.5% (w/w), based on the total weight of the composition as measured prior to precipitation and gel formation.

16. The method of claim 12, wherein the chitosan comprises from about 2.9-4.0% (w/w) and the albumin comprises from about 91-96.9% (w/w), based on the total weight of the composition as measured prior to precipitation and gel formation.

17. The method of claim 12, wherein the gel composition has a viscosity of from about 700 to about 250,000 Saybolt Seconds Universal at 16° C.

18. The method of claim 12, wherein the method is used for an application selected from the group consisting of head and neck surgical repairs, tracheal repairs, aerodigestive endoscopic repairs, endoscopic endonasal surgical repairs, iatrogenic esophageal perforation repairs, laparoscopic surgical repairs, lung repairs, colon repairs, repair of the tympanic membrane (ear drum), neurological repairs such as reattachment of severed nerves, anastomosis of vessels, urologic/gynecologic endoscopic pelvic repairs, orofacial surgical repairs, dental replacement, skin closure, thoracic surgical repairs, neurosurgery repairs, uterine closure and repairs after fibroidectomies and bladder surgery

19. The method of claim 18, wherein the method is used for skull base repair.

20. The method of claim 18, wherein the method is used for iatrogenic esophageal perforation repair.
